(19) United States
(12) Patent Application Publication
(10) Pub. No.: US 2002/0197289 A1
(43) Pub. Date: Dec. 26, 2002

(54) Compositions and methods for combating the appearance of ageing

(75) Inventors: Veronique Chevalier, Villecresnes (FR); Pascale Pelletier, Antony (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/102,729

(22) Filed: Mar. 22, 2002

(30) Foreign Application Priority Data
Mar. 23, 2001 (FR) 01 03957
Mar. 23, 2001 (FR) 01 03958
Mar. 23, 2001 (FR) 01 03959
Mar. 23, 2001 (FR) 01 03961
Mar. 23, 2001 (FR) 01 03962

(51) Int. Cl. A61K 7/00

(52) U.S. Cl. 424/401

(57) ABSTRACT

The present invention relates to a composition, especially a cosmetic composition, containing fibers and at least one anti-ageing active agent. The composition may be used to camouflage skin imperfections and to treat signs of ageing of the skin. The invention also relates to a composition containing fibers and at least one vitamin chosen from vitamin C, vitamin B3, vitamin B5, vitamin D, vitamin F, derivatives thereof, analogues thereof, precursors thereof and mixtures thereof, or from enzymes, steroids, and flavonoids.

### BACKGROUND OF THE INVENTION

1. Field of the Invention

The present invention relates to a composition, especially a topical cosmetic composition, containing fibers and at least one anti-ageing active agent. The invention further relates to the use of such compositions to camouflage imperfections in human skin and/or to treat signs of ageing of the skin. The invention also relates to a composition, preferably in a form for topical application, containing fibers and at least one vitamin chosen from vitamin C, vitamin B3, vitamin B5, vitamin D, vitamin F, derivatives thereof, analogues thereof, precursors thereof (i.e., derivatives, analogues and precursors of the five vitamins) and mixtures thereof, and to the uses of this composition, especially in cosmetics and dermatology. Similarly, the invention relates to compositions comprising fibers and at least one of an enzyme, steroid, and flavonaid, and to the uses of such compositions especially in cosmetics and dermatology, notably topically.

2. Discussion of the Invention

It is known that, during the ageing process, various signs appear on human skin that are very characteristic of this ageing, reflected especially by a change in the skin's structure and function. This ageing is of physiological nature but it may also be light-induced, that is to say that it may be due to the repeated exposure of the skin to light and, consequently, to the formation of oxygenated free radicals by the action of this light on the constituents of the skin.

The main clinical signs of ageing of the skin are especially the following: appearance of fine lines and then of deeper wrinkles which increase with age; disorganization of the “grain” of the skin, that is to say that the microrelief is less uniform and has an anisotropic nature; change in the complexion of the skin, which appears paler and yellower, which appears to be due essentially to a disorganization of the microcirculation (less haemoglobin in the papillary dermis); and the appearance of coloured marks at the surface, which is due to impairment of melanogenesis.

Another clinical sign of ageing is the dry and coarse appearance of the skin, and also a loss of firmness and tonicity of the skin.

It is known to treat these signs of ageing using cosmetic or dermatological compositions containing active agents capable of combating ageing, such as keratolytic agents, for instance α-hydroxy acids, β-hydroxy acids and retinoids, vitamins and any other agent capable of retarding the signs of ageing (sunscreens, ARLs, etc.). These active agents act on wrinkles and on the other signs of ageing differently depending on the active agent used, for example by removing the dead cells from the skin and by accelerating the process of cell renewal, by protecting skin against sunlight, by capturing free radicals or by moisturizing or nourishing the skin.

Thus, for example, ascorbic acid, or vitamin C, stimulates the synthesis of connective tissue and especially of collagen, reinforces the defenses of skin tissue against external attack such as ultraviolet rays and pollution, compensates for a vitamin E deficiency in the skin, depigments the skin and has a free-radical-scavenging function. It is thus an excellent candidate as a cosmetic or dermatological active agent for combating and/or preventing ageing of the skin.

Moreover, vitamins B3 and B5 can also act to treat or prevent ageing of the skin, especially by making the skin texture and/or the skin pore size uniform. They also have the property of removing the greasy appearance of the skin and of treating greasy skin.

Vitamin F makes it possible especially to combat dryness of the skin.

Vitamin D is an essential vitamin for preventing and treating mineralization defects in cartilage (rickets) and in bone (osteomalacia), and even certain forms of osteoporosis in the elderly. By topical application, vitamin D and its analogues make it possible especially to treat skin disorders such as, for example, psoriasis and the signs of ageing.

However, most anti-ageing active agents have the drawback of being effective for treating the signs of ageing of the skin only after they have been applied for a certain time. Now, it is increasingly sought to obtain an immediate effect of the active agents used, rapidly leading to a smoothing-out of wrinkles and fine lines and to the disappearance of fatigue marks and to fading out the visible signs of ageing, so as to obtain a smooth skin that radiates good health.

The inventors have found, surprisingly, that the combination of fibers and anti-ageing active agents makes it possible to obtain both an immediate and a long-term effect on the visible signs of ageing (e.g., fine lines, wrinkles, marks and dull complexion) and any other imperfection of treated keratin material and especially of the skin, while at the same time maintaining the skin's natural appearance. Here, the term “long-term effect” means that the composition will treat the skin, for example wrinkles of the skin, after some time of application, while the term “immediate” means that the composition will give an immediate effect of camouflage of the, e.g., wrinkles. In the invention compositions containing fibers and an anti-ageing active agent, it is believed that the fibers and perhaps the active agent give an immediate effect, and the active agent will give also an effect after some weeks or months of application (it is believed to act in the skin while fibers remain on the surface). In addition, the fibers provide good cosmetic properties: softness and comfort when applied to the skin, and ease of application.

One particular embodiment of the present invention is thus the cosmetic use of a cosmetic composition containing fibers and at least one anti-ageing active agent, to camouflage skin imperfections and/or to treat the signs of ageing of the skin.

Another subject of the invention is the use of fibers and of at least one anti-ageing active agent, for the preparation of a composition for topical application intended for treating ageing of the skin while at the same time camouflaging the signs of ageing.

The expression “topical application” means herein an external application to keratin materials, and the expression “keratin materials” means the skin, the scalp, the eyelashes, the eyebrows, the nails, the lips and mucous membranes. Keratin material affected herein is preferably human skin.

Another subject of the invention is a cosmetic process for treating the signs of ageing of keratin materials, and especially of the skin, characterized in applying to the keratin materials a cosmetic composition containing fibers and at least one anti-ageing active agent.

Since the composition used according to the invention is preferably intended for topical application, it preferably comprises a physiologically acceptable medium, that is to say a medium that is compatible with any keratin material such as the skin, including the scalp, the nails, mucous membranes, the eyes and the hair or any other area of body skin. It may especially constitute a cosmetic or dermatological composition.

The expression “anti-ageing active agent” means herein any active agent capable of treating or preventing any sign of ageing of the skin, including the scalp, the hair or mucous membranes (the lips), especially any agent capable of treating and/or preventing wrinkles and fine lines in the skin, of giving a clear complexion and of making the skin smooth.

Fibers

The fibers which may be used in the composition of the invention may be hydrophilic or hydrophobic fibers, of natural or synthetic, mineral or organic origin.

These fibers may be short or long, individual or organized, for example braided. They may have any shape or morphology and in particular may have a circular or polygonal (square, hexagonal or octagonal) cross section depending on the specific application envisaged. In particular, their ends are blunted and/or polished to prevent injury.

In particular, the fibers may have a length (L) ranging from 1 μm (0.001 mm) to 10 mm, preferably from 0.1 um to 5 mm and better still from 0.1 mm to 1.5 mm. Their cross section may be within a circle of diameter (D) ranging from 1 nm (0.001 μm) to 100 μm, preferably ranging from 1 nm (0.001 μm) to 50 um and better still from 5 um to 40 μm.

Preferably, the fibers used according to the present invention have a shape factor, i.e. a ratio L/D (length/diameter), ranging from 3.5 to 2,500, better still from 5 to 500 and even better still from 5 to 150.

The yarn count of the fibers is often given in denier or decitex. The denier is the weight in grams for 9 km of yam. The fibers used according to the invention preferably have a yarn count ranging from 0.15 to 30 denier and better still from 0.18 to 18 denier.

The shape factor, the yarn count and the morphology of the fibers are the three factors that are important for defining a fiber.

Useful fibers include those used in the manufacture of textiles and in particular silk, cotton, wool or flax fibers, cellulose fibers extracted in particular from wood, plants or algae, polyamide (Nylon®) fibers, modified cellulose (rayon, viscose or acetate, in particular rayon acetate) fibers, poly-p-phenyleneterephthalamide fibers, in particular Kevlar® fibers, acrylic fibers, in particular polymethyl methacrylate or poly (2-hydroxyethyl methacrylate) fibers, polyolefin fibers and in particular polyethylene or polypropylene fibers, glass, silica or aramid fibers, carbon fibers, in particular in the form of graphite, Teflon® fibers, insoluble collagen fibers, polyester, polyvinyl chloride, polyvinylidene chloride, polyvinyl alcohol, polyacrylonitrile, chitosan, polyurethane or polyethylene phthalate fibers, and fibers formed from a mixture of polymers such as those mentioned above, for instance polyamide/polyester fibers.

Examples of polyurethane fibers which may be mentioned are segmented polyurethane-urea) polymer fibers, belonging to the elastane class, and especially those sold under the name Lycra® by the company DuPont.

The resorbable synthetic fibers used in surgery may also be used, for instance the fibers prepared from glycolic acid and from caprolactone (Monocryl from the company Johnson & Johnson); resorbable synthetic fibers such as the copolymer of lactic acid and of glycolic acid (Vicryl from the company Johnson & Johnson); terephthalic polyester fibers (Ethibond from the company Johnson & Johnson) and stainless steel yarn (Acier from the company Johnson & Johnson).

Mixtures of the fibers mentioned above may also be used.

Moreover, the fibers may or may not be surface-treated and may be coated or uncoated. They may especially be coated and/or functionalized fibers, the term “functionalized” meaning that the fibers are surface-treated so as to modify their properties.

Useful coated fibers which may be used in the invention include polyamide fibers coated with copper sulphide for an antistatic effect (for example R-STAT from the company Rhodia) or another polymer allowing a particular organization of the fibers (specific surface treatment) or a surface treatment which induces colour/hologram effects (for example Lurex fiber from the company Sildorex).

The fibers can also be functionalized, that is to say be modified so as to have a specific function. This functionalization of the fibers can be carried out both on the fibers and in the fibers and by any method which makes it possible to attach a compound to the fibers or to trap it within the cavities formed by the geometry of the fibers. Mention may be made, as methods, of, for example, coating the fibers with an active principle; fixing, to the fibers, particles enclosing an active principle, such as nanocapsules or nanospheres; adsorption in the fibers; or fixing by chemical reaction. It is thus possible to use fibers having specific functional purposes, for example fibers which are stabilized against UV radiation by modification with chemical or physical sunscreens; fibers which have been rendered bactericidal or antiseptic by modification with preservatives or antibacterials; fibers which have been coloured by modification with colouring molecules; fibers which have been rendered keratolytic or desquamating by modification with keratolytic or desquamating agents; fibers which have been rendered hydrating by modification with hydrating agents or water-retaining polymers; fibers which have been rendered fragrant by modification with a fragrance; fibers which have been rendered analgesic or soothing by modification with an antiinflammatory or a soothing agent; or fibers which have been rendered resistant to perspiration by modification with an antiperspirant.

According to their properties, the fibers used according to the present invention may be introduced into an aqueous medium, an oily medium or into a powder, all within the skill of the ordinary worker in view of this disclosure.

The fibers which may be used according to the invention are preferably chosen from polyamide fibers, poly-p-phenyleneterephthalamide fibers and cotton fibers, and mixtures thereof. Their length preferably may range from 0.1 to 10 mm and more preferably from 0.1 to 1 mm, their mean diameter may range from 5 to 50 um and the shape factor preferably ranges from 5 to 150.

In particular, the polyamide fibers sold by Etablissements P. Bonte under the name Polyamide 0.9 dtex 0.3 mm, having a mean diameter of from 15 to 20 um, a yarn count of about 0.9 dtex (0.81 denier) and a length ranging from 0.3 mm to 1.5 mm, may be used. Poly-p-phenyleneterephthalamide fibers with a mean diameter of 12 um and a length of about 1.5 mm may also be used, such as those sold under the name Kevlar Floc by the company DuPont Fibers. These polyamide fibers are preferably introduced into an oily medium or introduced via a dry route into a powder.

It is also possible to use cotton fibers, for example those having a mean diameter of 20 um, a length of 0.3 mm and a shape factor of 15, such as those sold by the Institut Textile de France, the company Filature de Lomme, by the company Textiles des Dunes or by the company Velifil.

The fibers may be present in the composition according to the invention in an amount ranging, for example, from 0.01% to 50% by weight, preferably from 1% to 20% by weight and better still from 5% to 10% by weight relative to the total weight of the composition. A preferred amount of fibers is an amount that, in combination with the anti-ageing active agent(s) present, provide both an immediate and long term effect on the visible signs of ageing.

Anti-ageing Active Agents

As mentioned above, the anti-ageing active agent may be any active agent capable of treating or preventing any sign of ageing of the skin. One or more such agents may be used.

The active agents may be chosen, for example, from moisturizers, free-radical scavengers, keratolytic agents, vitamins, anti-elastase and anti-collagenase agents, protides, fatty acid derivatives, steroids, trace elements, bleaching agents, extracts of algae and of planktons, sunscreens, enzymes and coenzymes, flavonoids and ceramides, and mixtures thereof.

I. Useful moisturizers include sodium lactate; polyols, and in particular glycerol, sorbitol and polyethylene glycols; mannitol; amino acids; hyaluronic acid; lanolin; urea and mixtures containing urea, such as NMF (“Natural Moisturizing Factor”); petroleum jelly; and mixtures thereof.

II. Useful free-radical scavengers include phosphonic acid derivatives such as ethylenediaminetetra(methylenephosphonic acid), hexamethylenediaminetetra(methylenephosphonic acid), diethylenetriaminepenta(methylenephosphonic acid), and their salts and in particular their sodium salts, such as pentasodium ethylenediaminetetra(methylenephosphonic acid); ethylenediaminetetraacetic acid and its salts such as sodium salt; guanosine; superoxydismutase; tocopherol (vitamin E) and its derivatives (acetate); ethoxyquine; lactoferrin; lactoperoxidase and nitroxide derivatives; superoxide dismutases; glutathione peroxidase; plant extracts with free-radical-scavenging activity, such as the aqueous extract of wheatgerm sold by the company Silab under the reference Detoxiline; and mixtures thereof.

III. Useful keratolytic agents include α-hydroxy acids, especially acids derived from fruit, for instance glycolic acid, lactic acid, malic acid, citric acid, tartaric acid and mandelic acid, derivatives thereof and mixtures thereof; β-hydroxy acids, for instance salicylic acid and its derivatives such as 5-n-octanoylsalicylic acid or 5-n-dodecanoylsalicylic acid; α-keto acids, for instance ascorbic acid or vitamin C and its derivatives such as its salts, for instance sodium ascorbate, magnesium ascorbyl phosphate or sodium ascorbyl phosphate; its esters, for instance ascorbyl acetate, ascorbyl palmitate and ascorbyl propionate, or its sugars, for instance glycosylated ascorbic acid, and mixtures thereof; β-keto acids; retinoids, for instance retinol (vitamin A) and its esters, retinal, retinoic acid and its derivatives, and also the retinoids described in documents FR-A-2,570,377, EP-A-199 636, EP-A-325-540 and EP-A-402 072; and mixtures thereof.

IV. Useful vitamins, in addition to vitamins A, E and C indicated above, include vitamin B3 (or vitamin PP or niacinamide), vitamin B5 (or panthenol), vitamin D, vitamin F, derivatives, analogues and precursors of these vitamins and also those of vitamins A, E and C, for instance lycopenes or carotenes that are precursors of vitamin A, and mixtures thereof.

Vitamin B3, also known as vitamin PP, is a compound of formula:

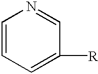

in which R may be —CONH₂ (niacinamide), —COOH (nicotinic acid or niacin), —CH₂OH (nicotinyl alcohol), —CO—NH—CH₂—COON (nicotinuric acid) or —CO—NH—OH (nicotinyl hydroxamic acid).

Vitamin B3 derivatives include nicotinic acid esters, such as tocopherol nicotinate; amides derived from niacinamide by substitution of the hydrogen groups of —CONH₂; products of reaction with carboxylic acids and amino acids; esters of nicotinyl alcohol and of carboxylic acids such as acetic acid, salicylic acid, glycolic acid or palmitic acid. Mention may also be made of the following derivatives: 2-chloronicotinamide, 6-methylnicotinamide, 6-aminonicotinamide, N-methylnicotinamide, N,N-dimethylnicotinamide, N-(hydroxymethyl)nicotinamide, quninolinic acid imide, nicotinanilide, N-benzylnicotinamide, N-ethylnicotinamide, nifenazone, nicotinaldehyde, isonicotinic acid, methylisonicotinic acid, thionicotinamide, nialamide, 2-mercaptonicotinic acid, nicomol and niaprazine.

Other vitamin B3 derivatives which may also be mentioned include its mineral salts such as the chlorides, bromides, iodides and carbonates, and its organic salts, such as the salts obtained by reaction with carboxylic acids such as acetate, salicylate, glycolate, lactate, malate, citrate, mandelate, tartrate, etc.

As vitamin B5, it is also possible to use panthenol or panthenyl alcohol or 2,4-dihydroxy-N(3-hydroxypropyl)-3,3-dimethylbutanamide, in its various forms: D-panthenol, DL-panthenol, and its derivatives and analogues, such as calcium pantothenate, pantethine, pantotheine, ethyl panthenyl ether, pangamic acid, pyridoxine and pantoyllactose, and natural compounds containing them such as royal jelly.

As vitamin D, mention may be made of 1α,25-dihydroxy vitamin D3 and its analogues, and also vitamin D analogues, such as those described in document WO-A-00/26167, such as, for example:

3-hydroxymethyl-5-{2-[3-(5-hydroxy-5- or 6-methylhexyl)-phenyl]-vinyl}-phenol,

3-[3-(5-hydroxy-1,5-dimethyl-hexyl)-phenoxymethyl]-5-hydroxymethyl-phenol,

6-[3-(3,4-bis-hydroxymethyl-benzyloxy)-phenyl]-2-methyl-hepta-3,5-dien-2-ol,

6-[3-(3,4-bis-hydroxymethyl-benzyloxy)-phenyl]-2-methyl-hexan-2-ol,

6-[3-(3,4-bis-hydroxymethyl-phenoxymethyl)-phenyl]-2-methyl-heptan-2-ol,

7-[3-(3,4-bis-hydroxymethyl-phenoxymethyl)-phenyl]-3-ethyl-octan-3-ol,

5-{2-[4-(5-hydroxy-5-methyl-hexyl)-phenyl]-vinyl or -ethyl}-benzene-1,3-diol,

5-{2-[3- or 4-(6-hydroxy-6-methyl-heptyl)phenyl]vinyl}-benzene-1,3-diol,

5-{2-[3- or 4-(6-hydroxy-6-methyl-heptyl)-phenyl]ethyl-benzene-1,3-diol,

2-hydroxymethyl-4-{2-[3- or 4-(5-hydroxy-5-methylhexyl)-phenyl]-vinyl-phenol,

2-hydroxymethyl-4-{2-[3- or 4-(6-hydroxy-6-methylheptyl)-phenyl]-vinyl}-phenol,

2-hydroxymethyl-4-{2-[3- or 4-(5-hydroxy-5-methylheptyl)-phenyl]-ethyl}-phenol,

2-hydroxymethyl-4-{2-[3- or 4-(6-hydroxy-6-methylheptyl)-phenyl]-ethyl}-phenol,

2-hydroxymethyl-5-(2-[4-(5-hydroxy-5-methyl-hexyl)phenyl]-vinyl-phenol,

6-[3-(3,4-bis-hydroxymethyl-benzyloxy)-phenyl12-methyl-heptan-2-ol,

4-[3-(5-hydroxy-1,5-dimethyl-hexyl)-phenoxymethyl]2-hydroxymethyl-phenol,

6-[3- or 4-[2-(3,4-bis-hydroxymethyl-phenyl)-vinyl]phenyl}-2-methyl-hexan-2-ol,

7-{4-[2-(3,4-bis-hydroxymethyl-phenyl)-vinyl]phenyl}-2-methyl-heptan-2-ol,

5-{2-[3-(6-hydroxy-6-methyl-heptyl)-phenyl]-1-methylvinyl-benzene-1,3-diol,

5-{2-[3-(5-hydroxy-5-methyl-hexyl)-phenyl]-vinyl}benzene-1,3-diol,

5-[3-(6-hydroxy-6-methyl-heptyl)-phenoxymethyl]benzene-1,3-diol,

5-{2-[3-(7-hydroxy-7-methyl-oct-1-enyl)-phenyl]vinyl}-benzene-1,3-diol,

5-{2-[3-(7-hydroxy-7-methyl-octyl)-phenyl]-vinyllbenzene-1,3-diol,

4-{2-[3-(6-hydroxy-6-methyl-heptyl)-phenyl]-vinyl)benzene-1,2-diol,

3-{2-[3-(6-hydroxy-6-methyl-heptyl)-phenyl]-vinyl}phenol,

6-{3-[2-(3,5-bis-hydroxymethyl-phenyl)-vinyl]phenyl}-2-methyl-hexan-2-ol,

3-{2-[3-(7-hydroxy-7-methyl-octyl)-phenyl]-vinyl}phenol,

7-{3-[2-(3,5-bis-hydroxymethyl-phenyl)-vinyl]phenyl-2-methyl-heptan-2-ol,

7-{3-[2-(3,4-bis-hydroxymethyl-phenyl)-vinyl]phenyl}-2-methyl-heptan-2-ol,

7-{3-[2-(4-hydroxymethyl-phenyl)-vinyl]-phenyl}2-methyl-heptan-2-ol,

4-{2-[3-(7-hydroxy-7-methyl-oct-1-enyl)-phenyl]vinyl}-benzene-1,2-diol,

7-[3-(3,4-bis-hydroxymethyl-phenylethynyl)-phenyl]2-methyl-heptan-2-ol,

5-{2-[3-(6-hydroxy-6-methyl-kept-1-enyl)-phenyl]vinyl}-benzene-1,3-diol,

5-{2-[3-(7-ethyl-7-hydroxy-non-1-enyl)-phenyl]vinyl)-benzene-1,3-diol,

5-{2-[3-(7-hydroxy-1-methoxy-1,7-dimethyl-octyl)phenyl]-vinyll-benzene-1,3-diol,

5-{2-[3-(6-hydroxy-1-methoxy-1,6-dimethyl-heptyl)phenyl]-vinyl}-benzene-1,3-diol,

5-{2-[3-(5-hydroxy-pentyl)-phenyl]-vinyl-benzene1,3-diol,

5-{2-[3-(5-hydroxy-6-methyl-heptyl)-phenyl]-vinyl}benzene-1,3-diol,

5-{2-[3-(6-hydroxy-7-methyl-octyl)-phenyl]-vinyl)benzene-1,3-diol,

5-{2-[3-(5-hydroxy-6-methyl-hept-1-enyl)-phenyl]vinyl}-benzene-1,3-diol,

5-{2-[3-(6-hydroxy-7-methyl-oct-1-enyl)-phenyl]vinyl}-benzene-1,3-diol,

5-{2-[3-(1,6-dihydroxy-1,6-dimethyl-heptyl)-phenyl]vinyl}-benzene-1,3-diol,

5-(2-[3-(6-hydroxy-1,6-dimethyl-kept-1-enyl)-phenyl]vinyl}-benzene-1,3-diol.

Vitamin F is a mixture of essential fatty acids, that is to say of unsaturated acids containing at least one double bond, such as linoleic acid or 9,12-octadecadienoic acid, and its stereoisomers, linolenic acid in α form (9,12,15-octadecatrienoic acid) or γ form (6,9,12-octadecatrienoic acid) and stereoisomers thereof, arachidonic acid or 5,8,11,14-eicosatetraenoic acid and its stereoisomers.

Vitamin F or analogues thereof such as mixtures of unsaturated acids containing at least one double bond and especially mixtures of linoleic acid, of linolenic acid and of arachidonic acid, or compounds containing them and especially oils of plant origin containing them such as, for example, jojoba oil, may be used in the composition of the present invention.

V. Useful anti-elastase agents include peptide derivatives and especially peptides from leguminous seeds such as those sold by Laboratoires Seriobiologiques de Nancy under the reference Parelastyl; the N-acylamino amide derivatives described in patent application FR-A-2,180,033, such as, for example, ethyl {2-[acetyl(3 trifluoromethylphenyl)amino]-3-methylbutyrylamino}acetate and {2-[acetyl-(3-trifluoromethylphenyl)amino]-3-methylbutyrylaminol acetic acid, and mixtures thereof. Anti-collagenase agents that may be mentioned include metalloprotease inhibitors, such as ethylenediamine acid (EDTA) and cysteine, and mixtures thereof.

VI. Useful protides include proteins (wheat or soybean protein), hydrolysates thereof, for instance those sold by the company Silab under the reference Tensine, and mixtures thereof.

VII. Useful fatty acid derivatives include polyunsaturated phospholipids including the essential fatty acid phospholipids from octopus, and mixtures thereof.

VIII. Useful steroids include DHEA or dehydroepiandrosterone, its biological precursors, its metabolites, and mixtures thereof. The expression “biological precursors” of DHEA especially means Δ5-pregnenolone, 17α-hydroxypregnenolone and 17α-hydroxypregnenolone sulphate. The expression “DHEA derivatives” means both its metabolic derivatives and its chemical derivatives. Metabolic derivatives that may especially be mentioned include Δ5-androstene-3,17-diol and especially 5-androstene-3β,17β-diol, Δ4-androstene-3,17-dione, 7-hydroxy DHEA (7α-hydroxy DHEA or 7β-hydroxy DHEA) and 7-keto-DHEA which is itself a metabolite of 7β-hydroxy DHEA. A preferred group is dehydroepiandrosterone, 5-pregnenolone, 17-hydroxypregnenolone, 17-hydroxypregnenolone sulphate, 5-androstene-3,17-diol, 4-androstene-3,17-dione, 7-hydroxy DHEA, 7-hydroxy DHEA, 7-keto-DHEA, and mixtures thereof.

IX. Useful trace elements include copper, zinc, selenium, iron, magnesium and manganese, and mixtures thereof.

X. Useful bleaching agents include any compound for treating or preventing age marks, that is to say any depigmenting compound which acts directly on the vitality of the epidermal melanocytes in which melanogenesis takes place and/or which interferes with one of the steps in the biosynthesis of melanin either by inhibiting one of the enzymes involved in melanogenesis or by becoming intercalated as a structural analogue of one of the chemical compounds in the synthetic chain of melanin, which chain may thus be blocked and bring about the depigmentation. Bleaching active agents that may be mentioned, for example, include kojic acid and its derivatives, hydroquinone and its derivatives such as arbutin and its esters; ellagic acid and its derivatives; plant extracts, and especially extracts of liquorice, of mulberry or of scutellaria; glutathione and its precursors; cysteine and its precursors; the compounds derived from aminophenol that are described in document WO-A-99/10318, such as, especially, N-ethyloxycarbonyl-4-aminophenol, N-ethyloxycarbonyl-O-ethyloxycarbonyl-4-aminophenol, N-cholesteryloxycarbonyl-4-aminophenol and N-ethylaminocarbonyl-4-aminophenol; and mixtures of these compounds.

XI. Useful extracts of algae include extracts of red or brown algae and, for example, the extract of brown algae from the Laminaria family, for instance the extracts from the species Laminaria digitata, and more particularly the product sold by the company CODIF under the name Phycosaccharides, which is a concentrated solution of an oligosaccharide obtained by controlled enzymatic depolymerization of membrane polysaccharides of a brown alga. It comprises a sequence of two uric acids: mannuronic acid and guluronic acid.

XII. Useful extracts of planktons include plankton in aqueous dispersion (CTFA name: Vitreoscilla Ferment) sold under the name Mexoryl SAH by the company Chimex.

XIII. Useful sunscreens include any chemical UVA and UVB screening agents or physical sunblocks that may usually be used in cosmetics.

UVB screening agents that may be mentioned, for example, include:

(1) salicylic acid derivatives, in particular homomenthyl salicylate and octyl salicylate; (2) cinnamic acid derivatives, in particular 2-ethylhexyl p-methoxycinnamate, sold by the company Givaudan under the name Parsol MCX;

(3) liquid β,β′-diphenylacrylate derivatives, in particular 2-ethylhexyl α-cyano-α,β′-diphenylacrylate or octocrylene, sold by the company BASF under the name Uvinul N539;

(4) p-aminobenzoic acid derivatives;

(5) 4-methylbenzylidenecamphor sold by the company Merck under the name Eusolex 6300;

(6) 2-phenylbenzimidazole-5-sulphonic acid sold under the name Eusolex 232 by the company Merck;

(7) 1,3,5-triazine derivatives, in particular:

2,4,6-tris[p-(2′-ethylhexyl-1′-oxycarbonyl)anilino]1,3,5-triazine sold by the company BASF under the name Uvinul T150, and

dioctylbutamidotriazone sold by the company Sigma 3V under the name Uvasorb HEB; (8) mixtures of these screening agents.

UVA screening agents that may be mentioned, for example, include:

(1) dibenzoylmethane derivatives, in particular, 4-(tert-butyl)-4′-methoxydibenzoylmethane sold by the company Givaudan under the name Parsol 1789;

(2) benzene-1,4-bis(3-methylidene-10-camphorsulphonic acid) optionally in partially or totally neutralized form, sold under the name Mexoryl SX by the company Chimex;

(3) benzophenone derivatives, for example:

2,4-dihydroxybenzophenone (benzophenone-1);

2,2′,4,4′-tetrahydrobenzophenone (benzophenone-2);

2-hydroxy-4-methoxybenzophenone (benzophenone-3) sold under the name Uvinul M40 by the company BASF;

2-hydroxy-4-methoxybenzophenone-5-sulphonic acid (benzophenone-4) and also its sulphonate form (benzophenone-5), sold by the company BASF under the name Uvinul MS40;

2,2′-dihydroxy-4,4′-dimethoxybenzophenone (benzophenone-6);

5-chloro-2-hydroxybenzophenone (benzophenone-7);

2,2′-dihydroxy-4-methoxybenzophenone (benzophenone8);

the disodium salt of 2,2′-dihydroxy-4,4′-dimethoxybenz:ophenone-5,5′-disulphonic diacid (benzophenone-9);

2-hydroxy-4-methoxy-4′-methylbenzophenone (benzophenone-10);

benzophenone-11; -2-hydroxy-4-(octyloxy)benzophenone (benzophenone-12);

(4) silane derivatives or polyorganosiloxanes containing a benzophenone group;

(5) anthranilates, in particular menthyl anthranilate sold by the company Haarman & Reimer under the name Neo Heliopan MA;

(6) compounds comprising, per molecule, at least two benzazolyl groups or at least one benzodiazolyl group, in particular 1,4-bis(benzimidazolyl)phenylene-3,3′,5,5′-tetrasulphonic acid and also its salts sold by the company Haarman & Reimer;

(7) silicon derivatives of N-substituted benzimidazolyl-benzazoles or of benzofuryl-benzazoles, and in particular:

2-[1-[3-[1,3,3,3-tetramethyl-1-[(trimethylsilyl) oxy]-disiloxanyl]propyl]-1H-benzimidazol-2-yl]benzoxazole;

2-[1-[3-[1,3,3,3-tetramethyl-1-[(trimethylsilyl) oxy]-disiloxanyl]propyl]-1H-benzimidazol-2-yl]benzothiazole;

2-[1-(3-trimethylsilanylpropyl)-1H-benzimidazol2-yl]benzoxazole;

6-methoxy-1,1′-bis(3-trimethylsilanylpropyl)-1H,1′H[2,2′]bibenzimidazolylbenzoxazole;

2-[1-(3-trimethylsilanylpropyl)-1H-benzimidazol2-yl]benzothiazole, which are described in patent application EP-A-1 028 120;

(8) triazine derivatives, and in particular 2,4-bis{[4-(2-ethylhexyloxy)-2-hydroxy]phenyl}-6-(4-methoxyphenyl)-1,3,5-triazine sold by the company Ciba Geigy under the name Tinosorb S, and 2,2′-methylenebis[6-(2H-benzotriazol-2-yl)-4-(1,1,3,3-tetramethylbutyl)phenol] sold by the company Ciba Geigy under the name Tinosorb M;

(9) benzotriazole silicones, which are described especially in patent application EP-A-0 392 883, in particular the benzotriazole silicone of formula:

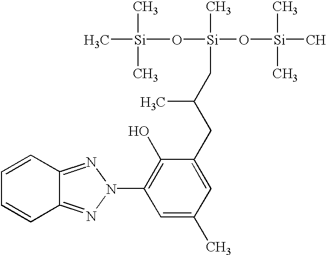

(10) mixtures thereof.

It is also possible to use a mixture of several of these screening agents and a mixture of UVB screening agents and UVA screening agents, and also mixtures with physical sunblocks.

Physical sunblocks that may be mentioned include titanium oxide (titanium dioxide in amorphous form or crystallized in rutile and/or anatase form), zinc oxide, iron oxide, zirconium oxide or cerium oxide, or mixtures thereof. These metal oxides may be in the form of particles of micrometric or nanometric size (nanopigments). In the form of nanopigments, the mean particle sizes range, for example, from 5 to 100 nm. Nanopigments are preferably used.

XIV. Useful enzymes that may be used include any enzyme of animal, microbiological (bacterial, fungal or viral) or synthetic origin (obtained by chemical or biotechnological synthesis), in pure crystalline form or in a form diluted in an inert diluent. Examples that may be mentioned are from among lipases, proteases, phospholipases, cellulases, peroxidases and especially lactoperoxidases, catalases and superoxide dismutases, or from among plant extracts containing the abovementioned enzymes, and mixtures thereof. They may be chosen, for example, from the product sold under the trade name “Subtilisine SP 554” by the company Novo Nordisk and from the product sold under the trade name “Lysoveg LS” by the company Laboratoires Serobiologiques de Nancy.

Coenzymes that may especially be used include ubiquinone or coenzyme Q10, which belongs to the family of alkylenated-chain benzoquinones, coenzyme R, which is biotin (or vitamin H), and mixtures thereof.

XV. Useful flavonoids that may be mentioned, for example, include isoflavonoids, which constitute a subclass of flavonoids, formed from a 3-phenylchroman skeleton which may comprise varied substituents and different levels of oxidation. The term “isoflavonoid” combines several classes of compounds, among which mention may be made of isoflavones, isoflavanones, rotenoids, pterocarpans, isoflavans, isoflavan-3-enes, 3-arylcoumarins, 3-aryl-4-hydroxycoumarins, coumestanes, coumaronochromones, a-methyldeoxybenzoins and 2-arylbenzofurans, and mixtures thereof. In this regard, reference will advantageously be made, for a complete review of isoflavonoids, their methods of analysis and their sources, to chapter 5 “Isoflavonoids” written by P. M. Dewick in The Flavonoids, edited by Harbone, pp. 125-157 (1988).

The isoflavonoids may be of natural or synthetic origin. The expression “natural origin” means an isoflavonoid in pure form or dissolved to various concentrations, obtained by various extraction processes from an element, generally a plant, of natural origin. The expression “synthetic origin” means an isoflavonoid in pure form or dissolved to various concentrations, obtained by chemical synthesis.

Isoflavonoids of natural origin are preferably used. Among these, mention may be made of: daidzin, genistin, daidzein, formononetin, cuneatin, genistein, isoprunetin and prunetin, cajanin, orobol, pratensein, santal, junipegenin A, glycitein, afrormosin, retusin, tectorigenin, irisolidone and jamaicin, and also analogues and metabolites thereof.

XVI. Useful ceramides that may be used include any type Of ceramide of natural or synthetic origin, for example of type II, of type III, of type IV, of type V or of type VI, and mixtures thereof.

Examples of ceramides that may be mentioned include N-oleoyldihydrosphingosine, N-stearoylphytosphingosine, N-α-hydroxybehenoyldihydrosphingosine, N-α-hydroxypalmitoyldihydrosphingosine, N-linoleoyldihydrosphingosine, N-palmitoyldihydrosphingosine, N-stearoyldihydrosphingosine and N-behenoyldihydrosphingosine, and mixtures thereof.

Mention may also be made of the product consisting of a mixture of glycoceramides, sold under the trade name Glycocer by the company Waitaki International Biosciences; the compounds described in documents EP-A-0 227 994 and WO-A-94/07844, such as, for example, Questamide H (bis(N-hydroxyethyl-Ncetyl)malonamide) sold by the company Quest, cetylic acid N-(2-hydroxyethyl)-N-(3-cetyloxy-2-hydroxypropyl)amide; N-docosanoyl-N-methyl-D-glucamine described in patent application WO-A-92/05764.

Mixtures of these ceramides may also be used.

The composition of the invention may comprise one or more anti-ageing active agents, of the same category or of different categories. The amount of active agent(s) in the composition depends on the active agent used and on the purpose of the composition. The active agent(s) must be present in an effective amount, that is to say in an amount that is sufficient to achieve the desired aim, which is to improve the appearance of the skin and to treat signs of ageing of the skin. The amount of active agent(s) in the composition may range, for example, from 0.0001% to 30% by weight, preferably from 0.01% to 20% by weight, better still from 0.1% to 15% by weight and even better still from 0.5% to 10% by weight relative to the total weight of the composition. A preferred amount is an amount that, in combination with the fibers present, provides both immediate and long term effects on a visible sign of ageing.

According to one particular embodiment, the anti-ageing active agent is a vitamin chosen from Vitamins C, B3, B5, D and F, and derivatives thereof. In another embodiment the agent is at least one of an enzyme, steroid and flavonoid.

Thus, a subject of the present invention is also a composition for topical application, containing the above-mentioned fibers and at least one vitamin chosen from vitamin C, vitamin B3, vitamin B5, vitamin D, vitamin F, derivatives thereof, precursors thereof and mixtures thereof, or at least one of an enzyme, steroid, and flavonoid. This composition may constitute a cosmetic or dermatological composition, preferably a cosmetic composition that can in particular constitute an anti-ageing composition.

Thus, the composition of the invention containing these vitamins may be used especially to combat and/or prevent ageing of the skin, especially by reducing wrinkles and fine lines, by attenuating or even eliminating marks appearing over time, by protecting the skin against UV radiation, by tonifying the skin, by regenerating the skin tissues, by giving a radiant complexion, and by making the skin texture and/or the skin pore size uniform.

Thus, a subject of the invention is also the cosmetic use of the composition as defined above for a cosmetic treatment of the skin in order to tonify it, regenerate it, smooth out fine lines in the skin, lighten the complexion, attenuate skin pigmentation marks, protect the skin against UV radiation, and/or make the skin texture and/or the skin pore size uniform.

Another subject of the invention is a cosmetic process for treating the signs of ageing of a keratin material, characterized in that it consists in applying to the keratin material a composition as defined above.

The physiologically acceptable medium for the compositions for topical application according to the invention may more particularly contain water and optionally a physiologically acceptable organic solvent chosen, for example, from lower alcohols containing from 1 to 8 carbon atoms and preferably 1 to 6 carbon atoms, for instance ethanol, isopropanol, propanol or butanol; polyethylene glycols containing from 6 to 80 ethylene oxides; polyols, for instance propylene glycol, isoprene glycol, butylene glycol, glycerol or sorbitol.

This medium may also be an anhydrous medium, especially an oily medium containing oils and/or fatty substances other than oils, as described below.

According to one preferred embodiment of the invention, the medium for the composition comprises water. This aqueous medium preferably has a pH that is compatible with the skin, preferably ranging from 3 to 8 and better still from 4.5 to 7.

When the composition comprises an aqueous or aqueous-alcoholic medium, it is possible to add a fatty phase (or oily phase) to this medium, so that the compositions of the invention are softer and more nourishing.

The oily phase usually contains at least one oil. As oils that may be used in the composition of the invention, mention may be made, for example, of:

hydrocarbon-based oils of animal origin, such as perhydrosqualene;

hydrocarbon-based oils of plant origin, such as liquid triglycerides of fatty acids containing from 4 to 10 carbon atoms, for instance heptanoic or octanoic acid triglycerides or alternatively, for example, sunflower oil, corn oil, soybean oil, marrow oil, grapeseed oil, sesame oil, hazelnut oil, apricot oil, macadamia oil, arara oil, sunflower oil, castor oil, avocado oil, caprylic/capric acid triglycerides, for instance those sold by the company Stearineries Dubois or those sold under the names Miglyol 810, 812 and 818 by the company Dynamit Nobel, jojoba oil or karite butter oil;

synthetic esters and synthetic ethers, especially of fatty acids, for instance oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate, isostearyl isostearate or isostearyl neopentanoate; hydroxylated esters such as isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;

linear or branched hydrocarbons of mineral or synthetic origin, such as volatile or non-volatile liquid paraffins, and derivatives thereof, petroleum jelly, polydecenes, and hydrogenated polyisobutene such as Parleam® oil;

fatty alcohols containing from 8 to 26 carbon atoms, for instance cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;

alkoylated and especially ethoxylated fatty alcohols such as oleth-12;

partially hydrocarbon-based and/or silicone-based fluoro oils, for instance those described in document JP-A-2 295 912. Examples of fluoro oils which may also be mentioned include perfluoromethylcyclopentane and perfluoro-1,3-dimethylcyclohexane, sold under the names “Flutec PC1®” and “Flutec PC3®” by the company BNFL Fluorochemicals; perfluoro-1,2-dimethylcyclobutane; perfluoroalkanes such as dodecafluoropentane and tetradecafluorohexane, sold under the names “PF 5050®” and “PF 5060®” by the company 3M, or alternatively bromoperfluorooctyl sold under the name “Foralkyl®” by the company Atochem; nonafluoromethoxybutane sold under the name “MSX 4518®” by the company 3M and nonafluoroethoxyisobutane; perfluoromorpholine derivatives, such as the 4-trifluoromethylperfluoromorpholine sold under the name “PF 5052®” by the company 3M;

silicone oils, for instance volatile or non-volatile polymethylsiloxanes (PDMSs) containing a linear or cyclic silicone chain, that are liquid or pasty at room temperature, especially cyclopolydimethylsiloxanes (cyclomethicones) such as cyclohexasiloxane; polydimethylsiloxanes comprising alkyl, alkoxy or phenyl groups, that are pendent or at the end of a silicone chain, these groups containing from 2 to 24 carbon atoms; phenylsilicones, for instance phenyltrimethicones, phenyldimethicones, phenyltrimethylsiloxydiphenylsiloxanes, diphenyldimethicones, diphenylmethyldiphenyltrisiloxanes, 2-phenylethyltrimethylsiloxysilicates and polymethylphenylsiloxanes;

mixtures thereof.

In the list of oils mentioned above, the expression “hydrocarbon-based oil” means any oil mainly comprising carbon and hydrogen atoms, and optionally ester, ether, fluoro, carboxylic acid and/or alcohol groups.

The other fatty substances that may be present in the oily phase are, for example, fatty acids containing from 8 to 30 carbon atoms, for instance stearic acid, lauric acid, palmitic acid and oleic acid; waxes, for instance lanolin, beeswax, carnauba wax or candelilla wax, paraffin wax, lignite wax or i microcrystalline waxes, ceresin or ozokerite, synthetic waxes such as polyethylene waxes, Fischer-Tropsch waxes; gums such as silicone gums (dimethiconol); silicone resins such as trifluoromethyl-C1-4-alkyldimethicone and trifluoropropyldimethicone; and silicone elastomers, for instance the products sold under the names “KSG” by the company Shin-Etsu, under the names “Trefil”, “BY29” or “EPSX” by the company Dow Corning or under the names “Gransil” by the company Grant Industries.

These fatty substances may be chosen in a varied manner by a person skilled in the art so as to prepare a composition having the desired properties, for example in terms of consistency or texture.

The compositions according to the invention may be in any form, including those conventionally used for topical application, and especially in the form of aqueous, aqueous-alcoholic or oily solutions, oil-in-water (O/W) or water-in-oil (W/O) or multiple (W/O/W or O/W/O) emulsions, aqueous or oily gels, liquid, pasty or solid anhydrous products, or dispersions of a fatty phase in an aqueous phase with the aid of spherules, these spherules possibly being polymer nanoparticles such as nanospheres and nanocapsules, or lipid vesicles of ionic and/or nonionic type. These compositions can be prepared according to the usual methods.

In addition, the compositions used according to the invention may be more or less fluid and may have the appearance of a white or coloured cream, an ointment, a milk, a lotion, a serum, a paste or a mousse. They may optionally be applied to the skin in the form of an aerosol. They may also be in solid form, and for example in the form of a stick.

According to one particular embodiment of the invention, the composition according to the invention is an emulsion. The proportion of the oily phase in the emulsion may range from 5% to 80% by weight and preferably from 5% to 50% by weight relative to the total weight of the composition. The oils, emulsifiers and co-emulsifiers used in the composition in emulsion form are chosen from those conventionally used in cosmetics or dermatology. The emulsifier and the co-emulsifier are generally present in the composition in a proportion ranging from 0.3% to 30% by weight and preferably from 0.5% to 20% by weight relative to the total weight of the composition. The emulsion may also contain lipid vesicles.

The emulsions generally contain at least one emulsifier chosen from amphoteric, anionic, cationic and nonionic emulsifiers, used alone or as a mixture. The emulsifiers are chosen in a suitable manner depending on the emulsion to be obtained (W/O or O/W emulsion). When the emulsion is a multiple emulsion, it generally comprises an emulsifier in the primary emulsion and an emulsifier in the external phase into which the primary emulsion is introduced.

As emulsifying surfactants that may be used for the preparation of the W/O emulsions, mention may be made, for example, of the alkyl esters or ethers of sorbitan, of glycerol or of sugars; silicone surfactants, for instance dimethicone copolyols such as the mixture of polyglyceryl-4 isostearate/cetyl dimethicone copolyol/hexyl laurate, sold under the name Abil WE 09® by the company Goldschmidt, the mixture of cyclomethicone and of dimethicone copolyol, sold under the names DC 5225 C and DC 3225 C by the company Dow Corning, and alkyldimethicone copolyols such as the laurylmethicone copolyol sold under the name “Dow Corning 5200 Formulation Aid” by the company Dow Corning and the cetyldimethicone copolyol sold under the name Abil EM 90® by the company Goldschmidt. It is also possible to add thereto one or more co-emulsifiers, which may be chosen advantageously from the group comprising esters of a branched-chain fatty acid and of polyol, and especially esters of a branched-chain fatty acid and of glycerol and/or of sorbitan, and, for example, polyglyceryl isostearate, such as the product sold under the name Isolan GI 34 by the company Goldschmidt, sorbitan isostearate, such as the product sold under the name Arlacel 987 by the company ICI, and the isostearate of sorbitan and of glycerol, such as the product sold under the name Arlacel 986 by the company ICI, and mixtures thereof.

For the O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alkyl ethers; sugar esters, for instance sucrose stearate, and mixtures thereof such as, for example, the mixture of glyceryl stearate and of PEG-100 stearate, sold under the name Aracel 165 by the company Uniqema.

Surfactant-free emulsions may also be prepared by using suitable compounds, for example polymers with emulsifying properties such as carbopol 1342 and the Pemulen products, and particles of ionic or nonionic polymers, more particularly particles of anionic polymer such as, especially, isophthalic acid or sulphoisophthalic acid polymers, and in particular copolymers of phthalate/sulphoisophthalate/glycol (for example diethylene glycol/phthalate/isophthalate/1,4-cyclohexanedimethanol; CTFA name: diglycol/CHDM/isophthalates/SIP copolymer) sold under the names “Eastman AQ polymer” (AQ35S, AQ38S, AQ55S and AQ48 Ultra) by the company Eastman Chemical.

The (cosmetic or dermatological) compositions of the invention may also contain adjuvants that are common in cosmetics or dermatology, such as hydrophilic or lipophilic gelling agents, hydrophilic or lipophilic active agents other than those mentioned above, preserving agents, antioxidants, solvents, fragrances, fillers, bactericides, odour absorbers, dyestuffs, salts and polymers (for example acrylates/dimethicone copolymer sold under the name KP-561 by Shin-Etsu, as dispersant). The amounts of these various adjuvants are those conventionally used in the field under consideration, and, for example, from 0.01% to 20% relative to the total weight of the composition. Depending on their nature, these adjuvants may be introduced into the fatty phase, into the aqueous phase and/or into lipid spherules.

Gelling agents that may be mentioned, for example, include carboxyvinyl polymers such as carbopols (carbomers) and Pemulens (acrylate/C₁₀-C₃₀-alkylacrylate copolymer); polyacrylamides such as, for example, the crosslinked copolymers sold under the names Sepigel 305 (CTFA name: polyacrylamide/C13-C14 isoparaffin/Laureth 7) or Simulgel 600 (CTFA name: acrylamide/sodium acryloyldimethyltaurate copolymer/isohexadecane/polysorbate 80) by the company>SEPPIC; 2-acrylamido-2-methylpropanesulphonic acid polymers and copolymers, that are optionally crosslinked and/or neutralized, for instance the poly(2-acrylamido-2-methylpropanesulphonic acid) sold by the company Hoechst under the trade name “Hostacerin AMPS” (CTFA name: ammonium polyacryldimethyltauramide); cellulose derivatives such as hydroxyethylcellulose; polysaccharides and especially gums such as xanthan gum; and mixtures thereof.

As fillers which may be used in the composition of the invention, mention may be made, for example, besides pigments, of silica powder; talc; polyamide particles and especially those sold under the name Orgasol by the company Atochem; polyethylene powders; microspheres based on acrylic copolymers, such as those based on ethylene glycol dimethacrylate/lauryl methacrylate copolymer sold by the company Dow Coming under the name Polytrap; expanded powders such as hollow microspheres and especially the microspheres sold under the name Expancel by the company Kemanord Plast or under the name Micropearl F 80 ED by the company Matsumoto; powders of natural organic materials such as crosslinked or noncrosslinked corn starch, i wheat starch or rice starch, such as the powders of starch crosslinked with octenyl succinate anhydride, sold under the name Dry-Flo by the company National Starch; silicone resin microbeads such as those sold under the name Tospearl by the company Toshiba Silicone; clays (bentone, laponite, saponite, etc.) and mixtures thereof. These fillers may be present in amounts ranging from 0% to 20% by weight and preferably from 1% to 10% by weight relative to the total weight of the composition.

The use of the invention compositions is within the skill of the ordinary artisan in view of this disclosure. For example, 0.1-5 g of invention composition may be applied to the skin once or more daily, for extended periods if desired.

The examples which follow serve to illustrate the invention without, however, being limiting in nature. The names are, depending on the case, the chemical names or the CTFA (International Cosmetic Ingredient Dictionary and Handbook) names and the amounts are given in percentages by weight, except where otherwise mentioned, based on total weight.

### EXAMPLE 1

### Triple Emulsion

| | |
|---|---|
| 1. Primary emulsion: | |
| Phase A: | |
| Abil WE 09 | 2.5% |
| Parleam oil | 17.5% |
| Polydimethylsiloxane | 4% |
| Phase B: | |
| Glycerol | 39% |
| Sequestering agent | 0.1% |
| Preserving agent | 0.8% |
| Demineralized water | 36.1% |
| 2. Triple emulsion: | |
| Phase A: | |
| Primary emulsion | 20.0% |
| Parleam oil | 5% |
| Retinol | 0.1% |
| Phase B: | |
| Hostacerin AMPS 0.5 | |
| Acrylate/C₁₀-C₃₀-alkylacrylate copolymer | |
| (Pemulen TR1) | 0.3% |
| Preserving agents | 1% |
| Demineralized water | 40% |
| Phase C: | |
| Triethanolamine | 0.3% |
| Demineralized water | 2% |
| Phase D: | |
| Hostacerin AMPS | 1.5% |
| Demineralized water | qs 100% |
| Phase E: | |
| Polyamide fibers (polyamide 0.9 dtex, | 5% |
| 0.3 mm - Paul Bonte company) | |

The triple emulsion is prepared in the following manner:

1. The primary emulsion is prepared by mixing together the constituents of phase A at room temperature, separately mixing together the constituents of phase B at room temperature, and slowly adding phase B to phase A with rapid stirring.

2. To prepare the triple emulsion, the various phases are prepared and phase A is then poured slowly into phase B with rapid stirring. Phase C is added thereto, then phase D and finally phase E at about 40° C. Stirring is continued until homogenization is complete.

A moisturizing cream capable of improving the radiance of the complexion and of fading out the signs of ageing is obtained.

### EXAMPLE 2

A composition similar to that of Example 1 may be prepared by replacing the retinol with glycolic acid.

### EXAMPLE 3

### O/W Emulsion

| Phase A: | |
|---|---|
| Hydrogenated polyisobutene | 5.5% |
| Isostearyl neopentanoate | 3.5% |
| PEG-20 stearate | 1% |
| Glyceryl stearate and PEG-100 stearate | 2% |
| (Arlacel 165) | |
| Cetyl alcohol | 0.5% |
| Stearyl alcohol | 0.5% |
| Stearic acid | 1% |
| Phase A′: | |
| Cyclomethicone | 11% |
| Cotton fibers | 3% |
| Phase B: | |
| Preserving agents | qs |
| Triethanolamine | 0.03% |
| Water | qs 100 |
| Phase C: | |
| Sepigel 305 | 1% |
| Parelastyl | 5% |

Procedure: Phase A is heated with stirring until homogeneous. After cooling, phase A′ is added. Phase B is heated with stirring, then B is poured into A with continued stirring. After cooling to 50° C., phase C is incorporated into the emulsion. A cream that is capable of fading out and of treating wrinkles and fine lines is obtained.

### EXAMPLE 4

### Fluid Emulsion

| | |
|---|---|
| Phase A | |
| N-Ethyloxycarbonyl-4-aminophenol | 0.5% |
| Mixture of oxyethylenated (20 EO) cetyl | 2% |
| alcohol and stearyl alcohol | |
| Oxyethylenated (60 EO) hydrogenated | 2.5% |
| castor oil | |
| Phase A′ | |
| Apricot oil | 5% |
| UV screening agents | 3.9% |
| Apricot oil | 5% |
| Cyclohexasiloxane | 10% |
| Polyethoxylated (20 EO) methylglucose | 2% |
| sesquistearate | |
| Phase B | |
| Preserving agents | 0.65% |
| Triethanolamine | 0.5% |
| Disodium EDTA | 0.05% |
| Glycerol | 5% |
| Demineralized water | qs: 100 |
| Phase C | |
| Gelling agents | 4.25% |
| Phase D | |
| Polyamide fibers (polyamide 0.9 dtex, | 5% |
| 0.3 mm - Paul Bonte company) | |

The composition is prepared in the following manner: phase A is heated to about 80° C. until completely dissolved, and is then introduced into phase B that has been preheated to the same temperature. Phase A′ is heated to about 80° C. and is then introduced into the mixture previously obtained, to form an O/W emulsion to which phases C and D are then added.

The emulsion obtained is fluid and makes it possible to fade out pigmentation marks on the face, the neckline and the hands.

### EXAMPLE 5

### W/O Emulsion

| | |
|---|---|
| Phase A | |
| Mixture of dimethicone copolyol and of | 20% |
| cyclomethicone (Q2-3225C from the company | |
| Dow Corning) | |
| Phenyltrimethicone (Dow Corning 556 fluid) | 4% |
| Plant oil | 3% |
| Cotton fibers | 1% |
| Phase B | |
| Glycerol | 23% |
| Propylene glycol | 6% |
| Sodium hydroxide | 1.8% |
| Citric acid | 1.2% |
| Ascorbic acid | 5% |
| Water | qs 100% |

The emulsion is prepared by separately preparing phases A and B, and introducing phase B into phase A with stirring.

A cream that is suitable for facial care, that is soft when applied and that affords immediate radiance of the complexion and fades out and smoothes out skin imperfections is obtained.

### EXAMPLE 6

### O/W Emulsion

| Oily phase | |
|---|---|
| Stearyl alcohol | 1% |
| Arlacel 165 | 2% |
| Cyclohexadimethylsiloxane | 10% |
| Aqueous phase | |
| vitamin B3 (niacinamide) | 2% |
| Glycerol | 5% |
| Carbomer | 0.2% |
| Xanthan gum | 0.2% |
| Sodium hydroxide | 0.01% |
| EDTA (sequestering agent) | 0.05% |
| Preserving agents | 0.2% |
| Aluminium starch octenylsuccinate | 3% |
| (Dry Flo from the company National Starch) | |
| Water | qs: 100% |
| Polyamide fibers (Polyamide 0.9 dtex, | |
| 0.3 mm - Paul Bonte company) | 8% |

The emulsion is prepared by separately preparing phases A and B, and introducing phase B into phase A with stirring, followed by addition of the fibers at a temperature of about 40° C. with stirring.

A cream that is capable of dissimulating and treating skin imperfections and also of making the skin texture and/or the skin pore size uniform is obtained.

### EXAMPLE 7

### Triple Emulsion

1. Primary emulsion:

Phase A:

Abil WE-09 2.5%

Polydimethylsiloxane 4%

Cyclopentadimethylsiloxane 17,5%

Phase B:

Glycerol 2%

Propylene glycol 20

Magnesium sulfate 0.8%

Preserving agent 0.2%

Demineralized water 53%

2. Triple emulsion:

Phase A:

Primary emulsion 25%

Cyclopentadimethylsiloxane 10%

Phase B:

Subtilisine SP554 0.1%

Carbopol 1342 0.3%

Carbopol 980 1%

Triethanolamine 0.3%

Glycerol 5%

Demineralized Water qsp 100%

Phase C:

Cotton fibers 1%

The triple emulsion is prepared in the following manner:

The primary emulsion is prepared by preparing separately phases A and B and introducing phase B into phase A while stirring. To prepare the triple emulsion, the various phases are prepared (phase B without triethanolamine and subtilisine), and phase A is then poured slowly into phaseB with rapid stirring. Triethanolamine and subtilisine are added thereto, then phase C. Stirring is continued until homogenization is complete.

A transparent cream capable of smoothing out the skin and lightening the complexion is obtained.

### EXAMPLE 8

### W/O Emulsion

Phase A

Dimethicone/dimethiconol 2.5%

Trifluoropropyl dimethicone 4%

Nylon 12 (Orgasol) 1.5%

Disteardimonium hectorite 3%

Polyamide fibers (NYLON-66) 12%

Cyclopentasiloxane 7%

Phase B

Mixture of dimethicone copolyol and of cyclomethicone

(Q2-3225C from the company Dow Coming) 10%

Acrylates/Dimethicone Copolymer (KP-561) 0.6%

Fragrance 0.1%

Phase C

Glycerol 0.3%

Ethanol 2.5%

Sodium chloride 5%

Subtilisine SP554 0.5%

Demineralized Water qs 100%

The emulsion is prepared by separately preparing and heating phases A (except Nylon-12 and fibers) and B (except fragrance), by cooling both phases, by introducing fragrance in phase B, by introducing phaseB into phaseA with stirring, by adding Nylon-12 and fibers, by preparing phase C except subtilisine, incorporating phase C into the preceding mixture and by adding subtilisine.

A white cream capable of attenuating and treating ageing signs is obtained.

### EXAMPLE 9

### Gel

Phase A

Sunscreen UVB 0.5%

Cyclohexasiloxane 5%

Polyamide fibers

(Polyamide 0.9 dtex, 0,3 mm-Ets Paul Bonte) 5%

Phase B

Preserving agents 0.65%

EDTA disodium 0.1%

Sodium hydroxide 0.11%

Glycerol 10%

Ethanol 2.5%

N-ethyloxycarbonyl-4-aminophenol 0.5%

LYSOVEG LS 1.5%

Carbomer 0.4%

Demineralized Water qsp 100%

This gel may be prepared by known methods, within the skill of the ordinary artisan in view of the disclosure herein to provide agel capable of attenuating and removing skin fine lines and skin pigmentation marks.

### EXAMPLE 10

### O/W Emulsion

Phase A

Glycerol Stearate 2.5%

PEG-8 stearate 2.5%

Stearic acid 1%

Octyldodecanol 8%

Mygliol 812 22%

DHEA 1%

Preserving agents 0.1%

Phase B

Triethanolamine 0.25%

Preserving agents 0.2%

Glycerol 5%

Demineralized Water qsp 100%

Phase C

Carbomer 0.3%

NaOH at 10% in water 0.25%

Demineralized Water 14.95%

Phase D

Polyamide fibers

(Polyamide 0.9 dtex, 0.3 mm-Ets Paul Bonte) 3%

Preparation: the phases A and B are heated separately at 75° C. Then phase B is incorporated in phase A by stirring and maintaining temperature. Then the mixture is cooled and phases C and D are added.

A white cream capable of attenuating and treating extrinsic and intrinsic ageing signs is obtained.

### EXAMPLE 11

### O/W Emulsion without Emulsifying Agent

Oily Phase

Oils of plant origin 12%

Sunscreen UVA 2%

Sunscreen UVB 4%

Cyclopentasiloxane 6%

Aqueous Phase:

Flavosterone SB (Ichimura Pharcos) 2%

EDTA disodium 0.05%

Copolymer diglycol/CHDM/Isophtahates/SIP

(Eastman AQ38S EASTMAN CHEMICAL) 2%

Glycerol 5%

Ethanol 10%

Nylon 12 (Orgasol) 0.2%

Demineralized Water qsp 100%

Polyamide fibers

(Polyamide 0.9 dtex, 0.3 mm-Paul Bonte) 2%

The composition is obtained by separately preparing aqueous phase and oily phase and by introducing oily phase into aqueous phase while stirring.

A comfortable cream capable of treating skin pigmentation marks, wrinkles and skin fine lines while keeping the natural appearance of the skin.

### EXAMPLE 12

### O/W Emulsion

Phase A

N-ethyloxycarbonyl-4-aminophenol 0.5%

Stearyle Heptanoate/Stearyle octanoate 5.5%

Oils of plant origin 11.6%

Sunscreen UVA 1.9%

Cyclopentasiloxane 3.7%

Sucrose Tristearate 2%

Polysorbate 61 1.35%

Stearic Acid 1%

Polyoxyethylene Hydrogenated castor oil (60 OE) 2.5%

Tocopherol acetate 0.5%

Phase B

FUJIFLAVONE P40 (Maruzen) 0.1%

Preserving agents 0.29%

Triethanolamine 0.5%

EDTA disodium 0.05%

Glycerol 3%

Demineralized Water qsp 100%

Polyamide fibers

(Polyamide 0.9 dtex, 0.3 mm-Paul Bonte) 6%

The composition is obtained by separately preparing phase A and phase B at 65° C., by cooling them to ambient temperature, by mixing phases A and B while stirring in high pressure homogeneizer, and by adding fibers.

A comfortable fluid for the face capable of attenuate and treating ageing signs, especially skin pigmentation marks, wrinkles and skin fine lines is obtained.

French patent applications 0103957, 0103958, 0103959 (Attorney docket OA01109), 0103961 (Attorney docket OA01111), and 0103962 (Attorney docket OA01112) all filed Mar. 23, 2001, are incorporated herein by reference, as are all documents, articles, patents, standards and references mentioned above.

1. A method for reducing visible signs of ageing both immediately and over a longer term, comprising applying a composition comprising fibers and an anti-ageing active agent to the keratin of a person in need thereof.

2. The method of claim 1, wherein said keratin is skin.

3. The method of claim 1, wherein the fibers have a length (L) ranging from 1 μm to 10 mm.

4. The method of claim 1, wherein the fibers have a cross section that is within a circle of diameter (D) ranging from 1 nm to 100 μm.

5. The method of claim 1, wherein the fibers have a shape factor (L/D) ranging from 5 to 150.

6. The method of claim 1, wherein the fibers have a yarn count ranging from 0.15 to 30 denier.

7. The method of claim 1, wherein the fibers are selected from the group consisting of silk, cotton, wool or flax fibers, cellulose fibers, polyamide (Nylon®) fibers, modified cellulose fibers, poly-p-phenyleneterephthalamide fibers, acrylic fibers, polyolefin fibers, glass, silica or aramid fibers, carbon fibers, Teflon® fibers, insoluble collagen fibers, polyester, polyvinyl chloride, polyvinylidene chloride, polyvinyl alcohol, polyacrylonitrile, chitosan, polyurethane or polyethylene phthalate fibers, fibers formed from a mixture of polymers, resorbable synthetic fibers, and mixtures thereof.

8. The method of claim 1, wherein the fibers are coated, functionalized, or coated and functionalized.

9. The method of claim 1, wherein the fibers are selected from the group consisting of polyamide fibers, poly-p-phenyleneterephthalamide fibers, cotton fibers, and mixtures thereof.

10. The method of claim 1, wherein the fibers are present in an amount ranging from 0.01% to 50% by weight relative to the total weight of the composition.

11. The method of claim 1, wherein the anti-ageing active agent is selected from the group consisting of moisturizers, free-radical scavengers, keratolytic agents, vitamins, anti-elastase and anti-collagenase agents, protides, fatty acid derivatives, steroids, trace elements, bleaching agents, extracts of algae and of planktons, sunscreens, enzymes and coenzymes, flavonoids and ceramides, and mixtures thereof.

12. The method of claim 1, wherein the anti-ageing active agent is a moisturizer selected from the group consisting of sodium lactate, polyols, mannitol, amino acids, hyaluronic acid, lanolin, urea and mixtures containing urea, petroleum jelly, and mixtures thereof.

13. The method of claim 1, wherein the anti-ageing active agent is a free-radical scavenger selected from the group consisting of phosphonic acid derivatives, ethylenediaminetetraacetic acid and its salts, guanosine, superoxydismutase, tocopherol and its derivatives, ethoxyquine, lactoferrin, lactoperoxidase, and nitroxide derivatives, superoxide dismutases, glutathione peroxidase, plant extracts with free-radical-scavenging activity, and mixtures thereof.

14. The method of claim 1, wherein the anti-ageing active agent is a keratolytic agent selected from the group consisting of a-hydroxy acids, especially acids derived from fruit, for instance glycolic acid, lactic acid, malic acid, citric acid, tartaric acid and mandelic acid and derivatives thereof; a-hydroxy acids, α-keto acids, P-keto acids; retinoids, and mixtures thereof.

15. The method of claim 1, wherein the anti-ageing active agent is a vitamin selected from the group consisting of vitamin A, vitamin C, vitamin E, vitamin B3, vitamin B5, vitamin D, vitamin F, and derivatives, analogues and precursors thereof, and mixtures thereof.

16. The method of claim 1, wherein the anti-ageing active agent is an anti-elastase agent or an anticollagenase agent selected from the group consisting of peptide derivatives, metalloprotease inhibitors, cysteine and N-acylamino amide derivatives, and mixtures thereof.

17. The method of claim 1, wherein the anti-ageing active agent is a protide selected from the group consisting of proteins, hydrolysates thereof, and mixtures thereof.

18. The method of claim 1, wherein the anti-ageing active agent is a fatty acid derivative selected from the group consisting of polyunsaturated phospholipids, and mixtures thereof.

19. The method of claim 1, wherein the anti-ageing active agent is a steroid selected from the group consisting of DHEA, its biological precursors, its metabolites, and mixtures thereof.

20. The method of claim 1, wherein the anti-ageing active agent is a trace element selected from the group consisting of copper, zinc, selenium, iron, magnesium, manganese, and mixtures thereof.

21. The method of claim 1, wherein the anti-ageing active agent is a bleaching agent selected from the group consisting of kojic acid and its derivatives; hydroquinone and its derivatives; arbutin and its esters; ellagic acid and its derivatives; extracts of liquorice, of mulberry or of scutellaria; glutathione and its precursors; cysteine and its precursors; N-ethyloxycarbonyl-4-aminophenol, N-ethyloxycarbonyl-O-ethyloxycarbonyl4-aminophenol, N-cholesteryloxycarbonyl-4-aminophenol, N-ethylaminocarbonyl-4-aminophenol; and mixtures thereof.

22. The method of claim 1, wherein the anti-ageing active agent is an extract of an alga from the Laminaria family.

23. The method of claim 1, wherein the anti-ageing active agent is a plankton in aqueous dispersion.

24. The method of claim 1, wherein the anti-ageing active agent is a sunscreen selected from the group consisting of chemical UVA or UVB screening agents and physical sunblocks, and mixtures thereof.

25. The method of claim 1, wherein the anti-ageing active agent is an enzyme selected from the group consisting of lipases, proteases, phospholipases, cellulases, peroxidases, catalases, superoxide dismutases, plant extracts containing these enzymes, and mixtures thereof.

26. The method of claim 1, wherein the anti-ageing active agent is a coenzyme selected from the group consisting of coenzyme Q10, coenzyme R, and mixtures thereof.

27. The method of claim 1, wherein the anti-ageing active agent is an isoflavonoid selected from the group consisting of isoflavones, isoflavanones, rotenoids, pterocarpans, isoflavans, isoflavan-3-enes, 3-arylcoumarins, 3-aryl4-hydroxycoumarins, coumestanes, coumaronochromones, α-methyldeoxybenzoins, 2-arylbenzofurans, and mixtures thereof.

28. The method of claim 1, wherein the anti-ageing active agent is a ceramide selected from the group consisting of N-oleoyldihydrosphingosine, N-stearoylphytosphingosine, N-α-hydroxybehenoyldihydrosphingosine, N-α-hydroxypalmitoyldihydrosphingosine, N-linoleoyldihydrosphingosine, N-palmitoyldihydrosphingosine, N-stearoyldihydrosphingosine, N-behenoyldihydrosphingosine, glycoceramides, and mixtures thereof.

29. The method of claim 1, wherein the amount of anti-ageing active agent(s) ranges from 0.0001% to 30% by weight relative to the total weight of the composition.

30. The method of claim 1, wherein the composition further comprises a physiologically acceptable medium comprising water.

31. The method of claim 1, wherein the composition is in the form of an emulsion.

32. A process for preparing a composition effective for treating the signs of ageing of a keratin material, comprising mixing fibers and at least one antiageing active agent together.

33. The process of claim 32, wherein said composition is in the form of an emulsion.

34. A composition comprising fibers and at least one vitamin selected from the group consisting of vitamin C, vitamin B3, vitamin B5, vitamin D and vitamin F, derivatives thereof, analogues thereof, precursors thereof and mixtures thereof.

35. The composition according to claim 34, wherein the fibers have a length L ranging from 1 μm to 10 mm.

36. The composition according to claim 34, wherein the fibers are present in an amount ranging from 0.01% to 50% by weight relative to the total weight of the composition.

37. The composition according to claim 34, wherein the vitamin is selected from the group consisting of ascorbic acid, its salts, its esters and its sugars; niacinamide, nicotinic acid, nicotinyl alcohol, nicotinuric acid, nicotinyl hydroxamic acid, and derivatives thereof; D-panthenol, DL-panthenol, and derivatives and analogues thereof, royal jelly; 1α,25-dihydroxyvitamin D3 and its analogues; vitamin D analogues; mixtures of unsaturated acids containing at least one double bond and plant oils containing them; and mixtures thereof.

38. The composition according to claim 34, wherein the amount of vitamin(s) ranges from 0.0001% to 30% by weight of active material relative to the total weight of the composition.

39. The composition according to claim 34, further comprising a physiologically acceptable medium comprising water.

40. The composition according to claim 34, in the form of an emulsion.

41. The composition according to claim 34, further comprising at least one active agent selected from the group consisting of moisturizers, free-radical scavengers, α-hydroxy acids, a-hydroxy acids, retinoids, anti-elastase agents, protides, fatty acid derivatives, steroids, trace elements, bleaching agents, extracts of algae, plankton, sunscreens, enzymes, co-enzymes, flavonoids and ceramides, and mixtures thereof.

42. The composition according to claim 34, wherein said composition is a cosmetic or dermatological composition.

43. A method for the cosmetic treatment of the skin in order to tonify it, regenerate it, smooth out fine lines in the skin, lighten the complexion, attenuate skin pigmentation marks and/or combat the harmful effects of UV radiation and/or remove the greasy appearance of the skin and/or make the skin texture and/or the skin pore size uniform, comprising applying the composition of claim 34 to the skin.

44. A composition comprising fibers and at least one anti-ageing active agent selected from the group consisting of enzymes, steroids and flavonoids.

45. The composition of claim 44, wherein the anti-ageing active agent is an enzyme selected from the group consisting of lipases, proteases, phospholipases, cellulases, peroxidases, catalases, superoxide dismutases, plant extracts containing these enzymes, and mixtures thereof.

46. The composition of claim 44, wherein the anti-ageing active agent is an isoflavonoid selected from the group consisting of isoflavones, isoflavanones, rotenoids, pterocarpans, isoflavans, isoflavan-3-enes, 3-arylcoumarins, 3-aryl4-hydroxycoumarins, coumestanes, coumaronochromones, α-methyldeoxybenzoins, 2-arylbenzofurans, and mixtures thereof.

47. The composition of claim 44, wherein the anti-ageing active agent is a steroid selected from the group consisting of dehydroepiandrosterone, 5-pregnenolone, 17-hydroxypregnenolone, 17-hydroxypregnenolone sulphate, 5-androstene-3, 17-diol, 4-androstene-3,17-dione, 7-hydroxy DHEA, 7-hydroxy DHEA, 7-keto-DHEA, and mixtures thereof.

48. The composition according to claim 44, wherein said composition is a cosmetic or dermatological composition.

49. The composition according to claim 44, wherein said composition is in the form of an emulsion.

50. A method for the cosmetic treatment of the skin in order to tonify it, regenerate it, smooth out fine lines in the skin, lighten the complexion, attenuate skin pigmentation marks and/or combat the harmful effects of UV radiation and/or remove the greasy appearance of the skin and/or make the skin texture and/or the skin pore size uniform, comprising applying the composition of claim 44 to the skin.
